(19) United States
(12) Patent Application Publication
(10) Pub. No.: US 2010/0222547 A1
(43) Pub. Date: Sep. 2, 2010

(54) Integrated Photoactive Peptides and Uses Thereof

(75) Inventors: Raghavan Rajagopalan, Solon, OH (US); William L. Neumann, St. Louis, MO (US); Richard B. Dorshow, St. Louis, MO (US)

Correspondence Address: GREENLEE WINNER AND SULLIVAN P C, 4875 PEARL EAST CIRCLE, SUITE 200, BOULDER, CO, 80301, US

(73) Assignee: MALLINCKRODT INC.

(21) Appl. No.: 12/528,631

(22) PCT Filed: Feb. 26, 2008
(86) PCT No.: PCT/US08/02463

§ 371 Date: Aug. 26, 2009

Related U.S. Application Data
(60) Provisional application No. 60/892,316, filed on Mar. 1, 2007.

(51) Int. Cl. C07K 7/08; C07K 7/06

(52) U.S. Cl. 530/326; 530/327; 530/329

(57) ABSTRACT

This invention is directed to the general method of transforming bioactive compounds of known structure and function into photoactive molecules such that the original biological activity is retained. The molecules resulting from the integration of two fundamental properties of photoactivity and biological function into a single molecular entity are hereinafter generally referred to as ‘integrated photoactive analogs’ or ‘integrated photoactive peptides or pseudopeptides.” The general method for the design of integrated photoactive analogs principally involves: (a) selecting a desired bioactive peptide or pseudopeptide; (b) identifying the region of the molecule that contains an aromatic or a heteroaromatic motif; and (c) either replacing said motif with a photoactive functional group of similar size, or modifying said motif to render it photoactive. Other aspects include photoactive analog compounds and photodiagnostic and phototherapeutic uses thereof.

### FIELD OF THE INVENTION

This invention relates generally to optical imaging, visualization, and phototherapy. Particularly, this invention relates to the structural integration of photoactive functional units into a bioactive targeting peptide or a pseudopeptide.

### BACKGROUND

Publications are referenced throughout the specification in parenthesis. Full citation corresponding to each reference is listed following the detailed description. The disclosures of these publications are herein incorporated by reference in their entireties in order to describe fully and clearly the state of the art to which this invention pertains.

Molecules absorbing, emitting, or scattering light in the visible, near-infra red (NIR), or long-wavelength (UV-A, >300 nm) region of the electromagnetic spectrum are useful for optical tomography, optical coherence tomography, fluorescence endoscopy, photoacoustic technology, sonofluorescence technology, light scattering technology, laser assisted guided surgery (LAGS), and phototherapy. The high sensitivity associated with fluorescence phenomenon parallels that of nuclear medicine, and permits visualization of organs and tissues without the negative effects of ionizing radiation. Targeted delivery to a particular site in the body of diagnostic and therapeutic agents (generally referred to as “haptens,” “effectors,” or “functional units”), such as fluorophores, photosensitizers, radionuclides, paramagnetic agents, and the like, continues to be of considerable demand in diagnosis, prognosis, and therapy of various lesions (Hassan et al., Licha et al., Shah et al., Vasquez et al., and Solban et al.). The conventional targeting method, referred to as “bioconjugate approach” or “pendant design” involves chemical attachment of these agents to bioactive carriers which target a particular site in the body. In the bioconjugate approach, the two units can exist and function independently wherein the functions of targeting and imaging/therapy may be separable. Bioactive carriers include small molecule drugs, hormones, peptidomimetics, enzyme inhibitors, receptor binders, receptor antagonists, receptor agonists, receptor modulators, DNA binders, transcription factors, inhibitors of the cell cycle machinery, transduction molecules, inhibitors of protein-protein interactions, inhibitors of protein-biomacromolecule interactions, macromolecular proteins, polysaccharides, polynucleotides, and the like. The bioconjugate approach has been explored extensively over the past several decades, and has met with moderate success, particularly in tumor detection, when medium and large size carriers (c.a. molecular weight>1000 Daltons) are employed (Licha et al. and Shah et al.). This is because attachment of dyes, drugs, metal complexes, or other effector molecules to macromolecular carriers such as antibodies, antibody fragments, or large peptides does not greatly alter the bioactive targeting properties; i.e., the bioconjugate is still able to bind to the receptor effectively. However, this approach does have some serious limitations in that the diffusion of high molecular weight bioconjugates to tumor cells is highly unfavorable, and is further complicated by the net positive pressure in solid tumors (Jain et al.). Furthermore, many dyes tend to form aggregates in aqueous media that lead to fluorescence quenching.

A need therefore exists for small photoactive molecules that also have bioactive targeting capabilities. However, a problem in designing small molecule bioconjugates is that the binding of a diagnostic or therapeutic agent to a targeted receptor is often observed to be severely compromised when the sizes of the diagnostic or therapeutic agent and the bioactive targeting carrier are similar (Hunter et al.). Thus, substituting a large functional unit such as a dye or a photosensitizer into small molecule drugs, peptides, pseudopeptides, or peptidomimetics presents a formidable challenge. In order to overcome this problem, methods (referred to as “integrated approach” or “internal bifunctional approach”) have been practiced wherein a radionuclide metal ion is incorporated into a steroid or morphine alkaloid framework such that the molecular topology of the original drug and the corresponding radionuclide mimic are very similar (Rajagopalan, U.S. Pat. No. 5,330,737; Rajagopalan, U.S. Pat. No. 5,602,236, and Horn et al.). In contrast to the bioconjugate approach described above, both functions of the integrated unit (e.g., targeting and imaging/therapy) are inseparable. The integrated approach is based on the principle that antibodies, enzymes, and receptors are multispecific and will bind to any molecule that is topologically similar to a natural antigen, substrate, or ligand. Previous work on steroid mimics confirm that integrating a metal ion into natural receptor ligands is a viable strategy for selective delivery of diagnostically and therapeutically useful radionuclides to target tissues (Horn, et al. and Skaddan et al.). This integrated design incorporates a single-atom isosteric substitution of a functional unit into a molecular framework. However, substituting a large functional unit such as a dye or a photosensitizer into small molecule drugs, peptides, pseudopeptides, or peptidomimetics presents a formidable challenge. While transformation of a nucleoside to fluorescent nucleoside has been previously reported (Miyata et. al.), the peak electronic spectra (absorption, excitation, and emission) remained in the UV region. In addition, this transformation is limited to this single nucleoside use.

### SUMMARY

Among the various aspects of the present invention, is the provision of an integrated photoactive analog of a peptide or pseudopeptide, methods of making the same, and diagnostic and therapeutic uses thereof.

In one aspect, the present invention is directed to a method of generating an integrated photoactive analog of a non-photoactive peptide or pseudopeptide. The method comprises replacing a non-photoactive functional group of the non-photoactive peptide or pseudopeptide with a photoactive functional group.

In another aspect, the invention is directed to a method of performing a diagnostic procedure on a patient. The method comprises administering an effective diagnostic amount of an integrated photoactive analog of a non-photoactive peptide or pseudopeptide to a patient.

In another aspect, the invention is directed to a method of performing a phototherapeutic procedure on a patient. The method comprises administering a therapeutically effective amount of an integrated photoactive analog of a non-photoactive peptide or pseudopeptide to a patient and irradiating the patient with a wavelength of light that causes photofragmentation of the molecule.

In still another aspect, the invention is directed to integrated photoactive analogs of non-photoactive peptides or pseudopeptides.

Other aspects and features of this invention will be in part apparent and in part pointed out hereinafter.

### DETAILED DESCRIPTION OF THE INVENTION

The present invention relates to the method of making and the use of integrated photoactive peptide or pseudo peptide analogs (hereinafter referred to as “integrated photoactive analogs” or simply “analogs”) of non-photoactive peptides or pseudopeptides wherein a non-photoactive functional group of the non-photoactive peptide or pseudopeptide is replaced with a photoactive moiety of similar size and molecular topology. The present invention also relates to methods of synthesizing an integrated photoactive analog by replacing a non-photoactive functional group with a photoactive moiety within a known non-photoactive peptide or pseudopeptide sequence. The integrated photoactive analog can be administered to a patient and utilized as a biooptical diagnostic contrast agent and/or a phototherapeutic agent. In one embodiment, the integrated photoactive analog is bioactive, wherein it targets a specific tissue, cell, receptor, and the like in a patient. In one example, the analog targets a diseased tissue, cell, receptor, and the like in a patient.

The integrated photoactive analogs of the present invention have absorption, excitation, and emission maximum wavelengths in the near-infra red (NIR) or visible spectrum of 350 nm or greater. This is beneficial for diagnostic or therapeutic treatment of patients since visible and NIR light is less likely to damage tissue when utilized in biooptical diagnostic and therapeutic procedures. In contrast, ultraviolet (UV) light that has a wavelength of less than 350 nm can result in tissue damage. Longer wavelength light of 350 nm or greater is also able to penetrate more deeply into tissues thereby permitting either diagnostic or therapeutic procedures to be conducted in the tissues of interest that are not reached by wavelengths that are less than 350 nm. In one embodiment, the integrated photoactive analogs have absorption, excitation, and emission maximum wavelengths between about 400 nm and about 900 nm.

Two general approaches for integrating structural and functional moieties into a single molecular analog include, (a) transforming a known bioactive peptide or pseudopeptide into an integrated photoactive analog; and (b) transforming a photoactive entity into an integrated photoactive analog that is bioactive. In either approach, the resulting molecules possess the fundamental properties of photoactivity and biological function. Depending on the structure and function, the integrated photoactive analogs of the present invention may be described as “integrated fluorophores,” “integrated chromophores,” “integrated photosensitizers,” and the like. The general method for the design of integrated photoactive analogs principally involves: (a) selecting a desired bioactive peptide or pseudopeptide; (b) identifying the region of the peptide or pseudopeptide that contains a replaceable moiety (e.g., aromatic, heteroaromatic, or aliphatic); and (c) either replacing said moiety with a photoactive functional group of similar size, or modifying said moiety to make it photoactive. The resulting integrated photoactive analog of the present invention is useful for both diagnostic and therapeutic applications.

The synthesis and use of integrated photoactive analogs may be performed in a variety of ways. In one embodiment, a peptide or a pseudopeptide with a known or desired structure and function is selected. For example, a selected photoactive peptide or pseudopeptide may target a specific tissue or cell of interest in a patient. A non-photoactive functional group within the molecular structure of the peptide or pseudopeptide is identified and replaced with a photoactive functional group to produce an integrated photoactive analog. The resulting integrated photoactive analog is administered to a patent in a diagnostically effective amount to detect the photoactive peptide or pseudopeptide within the patient. After a period of time has lapsed for the analog to bind to its target site, the whole body or a target tissue of a patient is exposed a light exhibiting a 350 to 1200 nm wavelength. In one example, the whole body or a target tissue of a patient is then exposed a light exhibiting a wavelength in the range of 400-900 nm. Light emanating from the patient as a result of the absorption and excitation of the integrated photoactive analog is then detected. By evaluating the location and strength of light emanating from the patient, a diagnosis may be made as a result of the targeting properties of the integrated photoactive analog.

The integrated photoactive analog can also be utilized to therapeutically treat a patient afflicted with a condition that exhibits a diseased tissue or cell that is targeted by the analog (e.g., a tumor, a fibrotic tissue, leukemia cell, and the like). After the integrated photoactive analog is administered to a patient, the analog targets and binds to the tissue, cell, receptor, or protein of interest. Light of an appropriate wavelength to photofragment/photoexcite the integrated photoactive analog into reactive species is administered to the patient in the area where the bound analog is located. The reactive species produced by the photofragmentation/photoexcitation of the integrated photoactive analog damages or kills diseased tissue or cells located in the proximity of the bound analog, thereby beneficially treating the patient's condition.

The development of an integrated photoactive analog involves selecting a suitable bioactive peptide or pseudopeptide that targets specific tissues, organs, lesions, cells, and the like. These include, but are not limited to, peptides and pseudopeptides that target tissues or organs such as brain, heart, liver, lung, or kidneys, diseased tissue such as cancerous tumors, leukemia cells, fibrotic epithelia, cystic fibrosis tissues, endometriotic tissues, and the like, receptors associated with a particular disease, such as tenascin C receptors or ST receptors, as well as infected or inflamed tissues. Non-limiting examples of peptides that target ST receptors that are associated with colon cancer are disclosed in U.S. Pat. No. 5,518,888, which is incorporated herein in its entirety. Other peptides or pseudopeptides, such as amino acid sequence ProLeuAlaGluIleAspGlyIleGluLeuThrTyr (SEQ ID NO: 1) have been found to bind to tenascin C, which is related to cystic fibrosis, metastasis of cancer, and myocardial viability (Schneider et al.). Bioactive peptides or pseudopeptides that may be used in the diagnosis and treatment of pathologic disorders such as cancer, atherosclerosis, restenosis, diabetic retinopathy, neovascular glaucoma, rheumatoid arthritis, endometriosis and other conditions related to angiogenesis are disclosed in U.S. Publication No. 20040053828, which is incorporated herein in its entirety. Examples of bioactive peptides or pseudopeptides include, but are not limited to, AlaAsnIleLysLeuSerValGlnMetLysLeu (SEQ ID NO: 2), SerValGlnMetLysLeu (SEQ ID NO: 3), IleLysLeuSerValGlnMetLysLeu (SEQ ID NO: 4), and AsnIleLysLeuSerValGlnMetLysLeu (SEQ ID NO: 5).

Fragments and/or derivatives of peptides and pseudopeptides that are also bioactive in targeting specific tissues, organs, receptors, etc. may also be modified or synthesized to photoactive molecules of the present invention. In accordance with conventional representation, the nomenclature used herein to define peptides and pseudopeptides is written such that, the N-terminal appears to the left and the C-terminal to the right in a given amino acid sequence.

Once a bioactive targeting peptide or pseudopeptide is selected, a non-photoactive moiety located on the peptide or pseudopeptide is identified and replaced with a photoactive moiety. Any moiety or portion of the peptide or pseudopeptide can be replaced by a photoactive moiety as long as the substitution does not result in substantial loss of biological activity or bioactive targeting properties of the resulting photoactive peptide or pseudopeptide. For example, a non-photoactive moiety on a peptide or pseudopeptide that targets a specific tissue, receptor, etc. can be replaced with a photoactive moiety so long as the resulting photoactive peptide or pseudopeptide also preferentially targets the specific tissue, receptor, etc.

In one embodiment, the non-photoactive moiety is an aromatic or heteroaromatic moiety located on the peptide or pseudopeptide which is replaced with a photoactive aromatic or heteroaromatic moiety. In one example, the non-photoactive aromatic or heteroaromatic functional group is a hydroxylphenyl group, an indolyl group, or a phenyl group. In another example, a peptide or pseudopeptide contains one or more amino acid residues having a non-photoactive aromatic or heteroaromatic moiety in its side chain such as tyrosine (Tyr/Y), tryptophan (Trp, W), phenylalanine (Phe/F), or histidine (His/H), which is replaced with a photoactive moiety. In another example, a non-photoactive aromatic or heteroaromatic moiety is replaced with an aromatic or heteroaromatic moiety having the same number of atoms in the ring structure as the non-photoactive moiety. In still another example, the non-aromatic or heteroaromatic moiety is replaced with a pyrazine, azulene, or azaazulene moiety.

In another embodiment, a non-photoactive side chain moiety of a non-aromatic or non-heteroaromatic amino acid residue within the peptide or pseudopeptide is substituted with a photoactive moiety. In one example, the non-aromatic or non-heteroaromatic moiety is replaced with a pyrazine, azulene, or azaazulene moiety.

In one embodiment, the photoactive moiety comprises a pyrazine moiety having the formula:

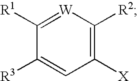

wherein R¹ to R³ are independently selected from the group consisting of hydrogen, alkyl, aryl, —OR⁴, —SR⁵, —NR⁶R⁷, —CN, —CO₂R⁸, —NO², —COR⁹, —CNR¹⁰R¹¹, —SOR¹², and —SO₂R¹³; W is N or —CR¹⁶; X is selected from the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, and —SO₂N(R²⁰)(CH₂)ₙ—; R⁴ to R²⁰ are independently selected from the group consisting of hydrogen, C1-C6 alkyl, C1 to C6 hydroxyalkyl, C1 to C6 alkoxyalkyl; and n varies from 0 to 10.

In another embodiment, the photoactive moiety comprises an azulene moiety having the formula:

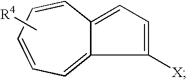

wherein X is selected from the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, and —SO₂N(R²⁰)(CH₂)ₙ—; R⁴ and R¹⁷ to R²⁰ are independently selected from the group consisting of hydrogen, C1-C6 alkyl, C1 to C6 hydroxyalkyl, C1 to C6 alkoxyalkyl; and n varies from 0 to 10.

In another embodiment, the photoactive moiety comprises an azaazulene moiety having the formula:

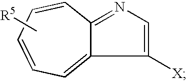

wherein X is selected from the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, and —SO₂N(R²⁰)(CH₂)ₙ—; R⁴ and R¹⁷ to R²⁰ are independently selected from the group consisting of hydrogen, C1-C6 alkyl, C1 to C6 hydroxyalkyl, C1 to C6 alkoxyalkyl; and n varies from 0 to 10.

In another embodiment, the integrated photoactive analog is a compound corresponding to Formula (A):

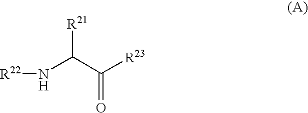

wherein R²¹ comprises a photoactive functional group, R²² is selected from the group consisting of hydrogen, an α-amino acid residue, and a sequence of two or more α-amino acid residues, and R²³ is selected from the group consisting of —OH, an α-amino acid residue, and a sequence of two or more α-amino acid residues

In one example, the compound of Formula (A) comprises a photoactive analog of a tyrosine, tryptophan, phenylalanine, or histidine residue having the structure:

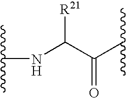

wherein R₂₁ comprises a side chain photoactive functional group.

Non-limiting examples of photoactive moieties of the present invention include, but are not limited to olefins, benzenes, naphthalenes, naphthoquinones, fluorenes, anthracenes, anthraquinones, phenanthrenes, tetracenes, naphthacenediones, pyridines, quinolines, quinazine, quinoxalines, quinidine, pteridine, isoquinolines, indoles, isoindoles, pyrroles, imidiazoles, oxazoles, thiazoles, pyrazoles, pyrazines, purines, benzimidazoles, furans, benzofurans, dibenzofurans, carbazoles, acridines, acridones, phenanthridines, thiophenes, benzothiophenes, dibenzothiophenes, xanthenes, xanthones, flavones, anthacylines; azulenes, and azaazulenes, indocyanines, benzoporphyrins, squaraines, corrins, coumarins, and cyanines. These photoactive moieties can be chemically converted into a biologically active photoactive analog (for example a receptor binding agent) by adding amino acid or peptide functional groups onto the photoactive moiety that cause the resulting photoactive analog to possess bioactivity or biological targeting properties.

The photoactive moieties of the present invention further include reactive species (or intermediates) useful in phototherapeutic procedures. Phototherapeutic moieties include, but are not limited to free radicals, carbenes, nitrenes, singlet oxygen, and the like. Examples of Type I photoreactive moieties that can be incorporated into a peptide or pseudopeptide for the purpose of synthesizing a phototherapeutic analog include, but are not limited to, azides, azo compounds, diazo compounds, sulfenates, thiadiazoles, peroxides, and the free radical or reactive intermediate formed upon irradiation. Examples of Type II photoreactive moieties that can be incorporated into a peptide or pseudopeptide for the purpose of synthesizing a phototherapeutic analog include, but are not limited to, phthalocyanines, porphyrins, extended porphyrins, and benzoporphyrins. This would be accomplished by chemically converting the phthalocyanine, porphyrin, extended porphyrin, and/or benzoporphyrin system to a biologically active substance (for example a receptor binding agent). This can be performed by adding functional groups onto the moiety that cause the resulting peptide or pseudopeptide to possess bioactivity or biological targeting properties.

In one embodiment, a bioactive peptide or pseudopeptide of the present invention comprises both a photoactive moiety and a photoreactive moiety.

Once an integrated photoactive analog has been created, the analog is administered to an individual. An appropriate amount of time is given for the analog to bind to the target tissue or cell, or the like in the patient. It will be understood that the administration of the compounds and compositions of the present invention is determined by the attending physician within the scope of sound medical judgment. The specific effective dose level for any particular patient depends upon a variety of factors including the disorder being treated, the severity of the disorder; activity of the specific compound employed; the specific composition employed, age, body weight, general health, sex, diet of the patient. The detection of the integrated photoactive analog is achieved by optical fluorescence, absorbance, or light scattering methods known in the art using invasive or non-invasive probes such as endoscopes, catheters, ear clips, hand bands, head bands, surface coils, finger probes, and the like (Muller et al.). The imaging can be achieved using planar imaging, optical tomographic, optical coherence tomographic, endoscopic, photoacoustic, sonofluorescent, confocal microscopic, or light scattering devices known in the art.

Similar to the diagnostic procedure described above, the integrated photoactive analog can be administered to an individual for therapeutic purposes. After administering the integrated photoactive analog to a patient, an appropriate amount of time is given for the analog to bind to the target tissue or cell, or the like in the patient. The patient may be optionally imaged as described above to determine the location where the analog is bound within the patient. Once the analog is determined to be bound to the targeted site or sites, the patient is irradiated with a wavelength and intensity of light sufficient to cause photofragmentation of the integrated photoactive analog. The photofragmentation typically results in homolytic cleavage of the analog, resulting in the generation of free radical intermediates. The generated free radicals then damage diseased tissues or cells of the targeted site(s) to which the integrated photoactive analog had bound, thereby therapeutically treating the condition of the patient.

In one embodiment, the non-photoactive peptide is a ST (heat sensitive bacterioenterotoxin) receptor binding sequence (Waldman, U.S. Pat. No. 5,518,888) represented by Formula 1:

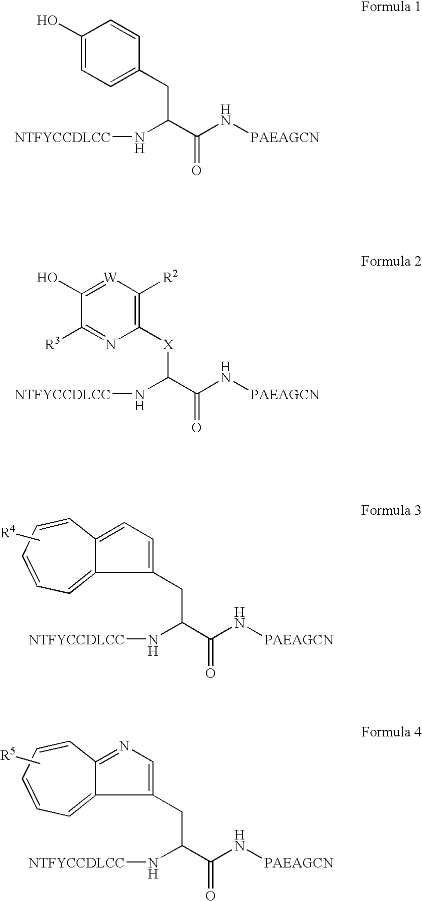

The non-photoactive peptide sequence of Formula 1, AsnThrPheTyrCysCysAspLeuCysCysTyrProAlaGluAlaGlyCysAsn (SEQ ID NO: 6), comprises a tyrosine residue that contains a non-photoactive hydroxyphenyl moiety in its side chain. The hydroxyphenyl moiety of the tyrosine residue is replaced with either a pyrazine (Formula 2), AsnThrPheTyrCysCysAspLeuCysCysXaaProAlaGluAlaGlyCysAsn (SEQ ID NO: 7); azulene (Formula 3), AsnThrPheTyrCysCysAspLeuCysCysXaaProAlaGluAlaGlyCysAsn (SEQ ID NO: 8); or azaazulene (Formula 4), AsnThrPheTyrCysCysAspLeuCysCysXaaProAlaGluAlaGlyCysAsn (SEQ ID NO: 9) photoactive moiety. The resulting analogs of Formulas 2-4 are photoactive, wherein R¹ to R³ are independently electron donating or electron withdrawing groups such as hydrogen, alkyl, aryl, —OR⁶, —SR⁷, —NR⁸R⁹, —CN, —CO₂R¹⁰, —NO₂, —COR¹¹, —CNR¹²R¹³, —SOR¹⁴, —SO₂R¹⁵, and the like. W is —N or —CR¹⁶. X is a spacer selected form the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, and —SO₂N(R²⁰)(CH₂)ₙ—; n varies from 0 to 10. R⁴ to R²⁰ are independently selected from the group consisting of hydrogen, C1-C6 alkyl, C1 to C6 hydroxyalkyl, and C1 to C6 alkoxyalkyl. The integrated photoactive peptides of this embodiment are useful for diagnosis, prognosis, and phototherapy of colorectal cancer.

In another embodiment, the non-photoactive peptide is a tenascin C binding sequence (Edelberg et al. and Schneider et al.) represented by Formula 5:

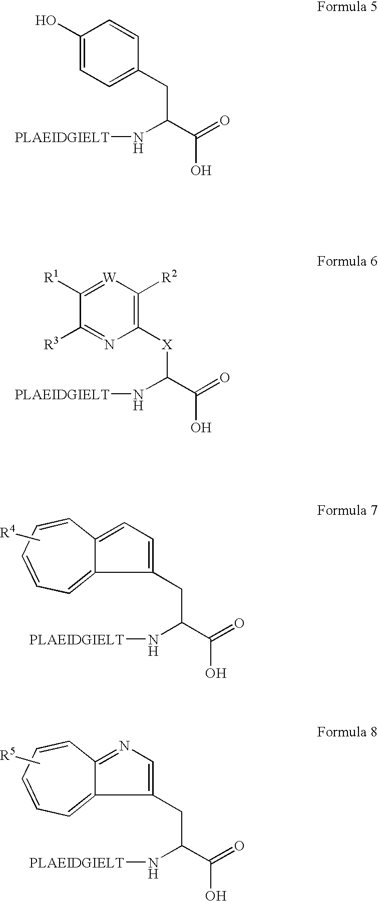

The non-photoactive peptide sequence of Formula 5, ProLeuAlaGluIleAspGlyIleGluLeuThrTyr (SEQ ID NO 10), comprises a tyrosine residue that contains a non-photoactive hydroxyphenol moiety in its side chain. The non-photoactive hydroxyphenol moiety is replaced with either a pyrazine (Formula 6), ProLeuAlaGluIleAspGlyIleGluLeuThrXaa (SEQ ID NO 11); azulene (Formula 7), ProLeuAlaGluIleAspGlyIleGluLeuThrXaa (SEQ ID NO 12); or azaazulene (Formula 8), ProLeuAlaGluIleAspGlyIleGluLeuThrXaa (SEQ ID NO 13) photoactive moiety. The resulting analogs of Formulas 6-8 are photoactive, wherein R¹ to R³ are independently electron donating or electron withdrawing groups such as hydrogen, alkyl, aryl, —OR⁶, —SR⁷, —NR⁸R⁹, —CN, —CO₂R¹⁰, —NO₂, —COR¹¹, —CNR¹²R¹³, —SOR¹⁴, —SO₂R¹⁵, and the like. W is —N or —CR¹⁶. X is a spacer selected form the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO₂(CH₂)ₙ—, SO₂(CH₂)ₙ—, and —SO₂N(R²⁰)(CH₂)ₙ—; n varies from 0 to 10. R⁴ to R²⁰ are independently selected from the group consisting of hydrogen, C1-C6 alkyl, C1 to C6 hydroxyalkyl, and C1 to C6 alkoxyalkyl. The integrated photoactive peptides of this embodiment are useful for the assessment of myocardial viability and cystic fibrosis.

In another embodiment, the non-photoactive peptide targets endometriotic tissue and has sequence (Nothick and Mayo et al.) represented by Formula 9:

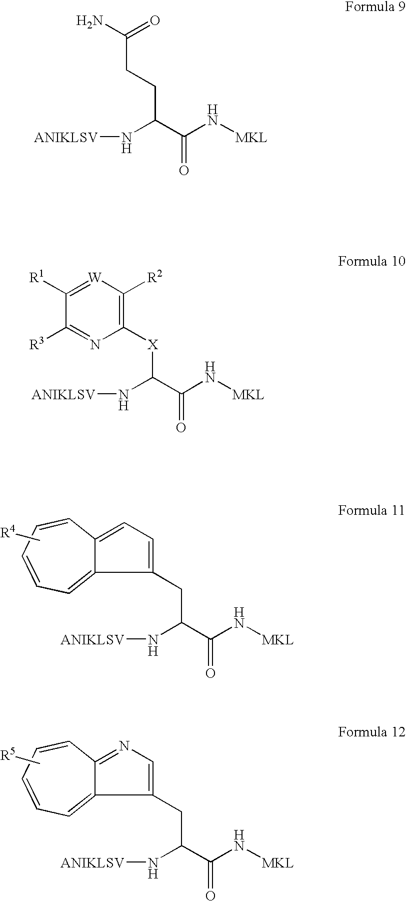

The non-photoactive peptide sequence of Formula 9, AlaAsnIleLysLeuSerValGlnMetLysLeu (SEQ ID NO 14), comprises a glutamine residue that contains a non-photoactive aliphatic group in its side chain. The non-photoactive aliphatic group is replaced with either a pyrazine (Formula 10), AlaAsnIleLysLeuSerValXaaMetLysLeu (SEQ ID NO 15); azulene (Formula 11), AlaAsnIleLysLeuSerValXaaMetLysLeu (SEQ ID NO 16); or azaazulene (Formula 12), AlaAsnIleLysLeuSerValXaaMetLysLeu (SEQ ID NO 17); photoactive moiety. The resulting analogs of Formulas 10-12 are photoactive, wherein R¹ to R³ are independently electron donating or electron withdrawing groups such as hydrogen, alkyl, aryl, —OR⁶, —SR⁷, —NR⁸R⁹, —CN, —CO₂R¹⁰, —NO₂, —COR¹¹, —CNR¹²R¹³, —SOR¹⁴, —SO₂R¹⁵, and the like. W is —N or —CR¹⁶. X is a spacer selected form the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, and —SO₂N(R²⁰)(CH₂)ₙ—; n varies from 0 to 10. R⁴ to R²⁰ are independently selected from the group consisting of hydrogen, C1-C6 alkyl, C1 to C6 hydroxyalkyl, and C1 to C6 alkoxyalkyl. The integrated photoactive peptides of this embodiment are useful for diagnosis, prognosis, and phototherapy of endometriosis.

In another embodiment, the non-photoactive peptide targets leukemia cells and has sequence (Jaalouk et al.) represented by Formula 13:

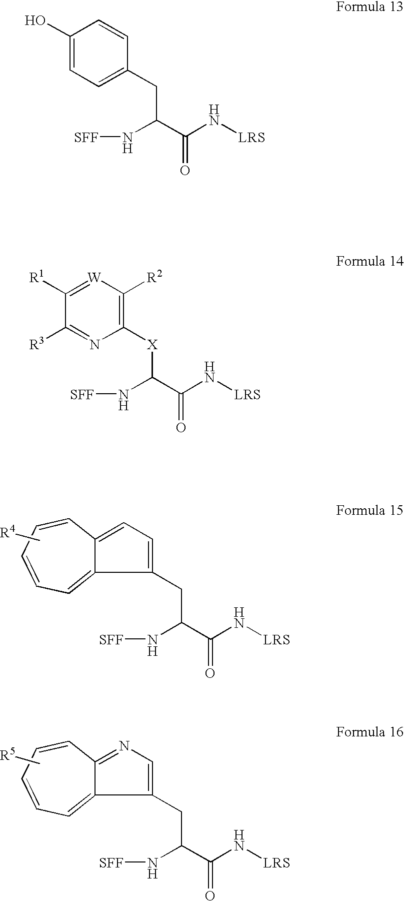

The non-photoactive peptide sequence of Formula 13, SerPhePheTyrLeuArgSer (SEQ ID NO: 18), comprises a tyrosine residue that contains a non-photoactive hydroxyphenyl group in its side chain. The non-photoactive hydroxyphenyl group is replaced with either a pyrazine (Formula 14), SerPhePheXaaLeuArgSer (SEQ ID NO: 19); azulene (Formula 15), SerPhePheXaaLeuArgSer (SEQ ID NO: 20); or azaazulene (Formula 16), SerPhePheXaaLeuArgSer (SEQ ID NO: 21); photoactive moiety. The resulting photoactive analogs of Formulas 14-16 are photoactive, wherein R¹ to R³ are independently electron donating or electron withdrawing groups such as hydrogen, alkyl, aryl, —OR⁶, —SR⁷, —NR⁸R⁹, —CN, —CO₂R¹⁰, —NO₂, —COR¹¹, —CNR¹²R¹³, —SOR¹⁴, —SO₂R¹⁵, and the like. W is —N or —CR¹⁶. X is a spacer selected form the group consisting of —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, and —SO₂N(R²⁰)(CH₂)ₙ—; n varies from 0 to 10. R⁴ to R²⁰ are independently selected from the group consisting of hydrogen, C1-C6 alkyl, C1 to C6 hydroxyalkyl, and C1 to C6 alkoxyalkyl. The integrated photoactive peptide of this embodiment is useful for diagnosis, prognosis, and phototherapy of leukemia.

Synthesis of Photoactive Derivatives

The synthesis of pyrazine, azulene, and azaazulene derivatives and the integrated photoactive analogs derived therefrom can typically be prepared by the Strecker process or other amino acid syntheses known in the art (Wentroup et al., Nozoe et al., and Schneider et al.). The synthesis of integrated photoactive analogs of the present invention can be accomplished by solution phase or automated solid phase peptide synthesis methods known in the art (Bodansky et al.). The solid phase method described in detail in the forthcoming examples generally employs fluorenylmethoxycarbonyl (Fmoc)-protected amino acids in a commercial peptide synthesizer (e.g. Applied Biosystems Model 432A SYNERGY Peptide Synthesizer). Each peptide cartridge contains Wang resin conjugated with Fmoc-amino acids with additional side chain protecting group, if necessary.

Formulation

The integrated photoactive agents of the present invention can be formulated for enteral (oral or rectal), parenteral, topical, transdermal, or subcutaneous administration. Topical, transdermal, and cutaneous delivery can also include aerosols, creams, gels, emulsions, solutions, or suspensions. Delivery into and through the skin can be enhanced in accordance with known methods and agents such as transdermal permeation enhancers, for example, “azone”, N-alkylcyclic amides, dimethylsulfoxide, long-chained aliphatic acids (C₁₀), etc. (Gennaro).

The method for preparing pharmaceutically acceptable formulations can be accomplished according to methods known in the art (Gennaro). A formulation is prepared using any of the integrated photoactive agents, along with pharmaceutically acceptable buffers, surfactants, excipients, thixotropic agents, flavoring agents, stabilizing agents, or skin penetration enhancing agents. If the inventive compound is water soluble, a solution in physiological saline may be administered. If the compound is not water soluble, the compound can be dissolved in a biocompatible oil (e.g., soybean oil, fish oil, vitamin E, linseed oil, vegetable oil, glyceride esters, long-chained fatty esters, etc.) and emulsified in water containing surface-active compounds (e.g., vegetable or animal phospholipids; lecithin; long-chained fatty salts and alcohols; polyethylene glycol esters and ethers; etc.), and administered as a topical cream, suspension, water/oil emulsion, or water/oil microemulsion.

The integrated photoactive agents may also be encapsulated into micelles, liposomes, nanoparticles, shell cross-linked nanoparticles, dendrimers, dendrons, microcapsules, or other organized microparticles, and administered by any of the routes described previously. The integrated photoactive agents may also be chemically conjugated to nanoparticles, shell cross-linked nanoparticles, dendrimers or dendrons for the purpose of simultaneously effecting an integrated photonic effect and a multivalent biological effect. These formulations may enhance stability of said agents in vivo. Encapsulation methods include detergent dialysis, freeze drying, film forming, or injection (Janoff et al.). The method of making liposomes and encapsulating various molecules within them are well known in the art (Braun-Falco et al. and Lasic et al.).

Dosage

The compositions comprising the integrated photoactive analogs of the present invention may be administered in a single dose or in many doses to achieve the effective diagnostic or therapeutic objective. After administration, the integrated photoactive analog accumulates at a target tissue, and the selected target site is exposed to light with a sufficient power and intensity to render a diagnosis and/or treatment. Such doses may vary widely depending upon the particular integrated photoactive analog employed, the organs or tissues to be examined, the equipment employed in the clinical procedure, the efficacy of the treatment achieved, and the like. The dose of the compound may vary from about 0.1 mg/kg body weight to about 500 mg/kg body weight, typically from about 0.5 to about 2 mg/kg body weight. For parenteral administration, a sterile solution or suspension comprises the integrated photoactive agent in a concentration range from about 1 nM to about 0.5 M. In another example, the sterile solution or suspension comprises the integrated photoactive agent in a concentration range from about 1 μM to about 10 mM.

Although the present invention can be beneficially utilized in the form of small molecules, the methodology is also applicable to any bioactive molecule, large or small. The present invention is useful for various biomedical optics applications including, but are not limited to, planar imaging, optical tomography, optical coherence tomography, endoscopy, photoacoustic technology, sonofluorescence technology, light scattering technology, laser assisted guided surgery (LAGS), confocal microscopy, dynamic organ function monitoring, and phototherapy.

### ABBREVIATIONS AND DEFINITIONS

To facilitate understanding of the invention, a number of terms are defined below:

The amino acid notations used herein for the twenty genetically encoded α-amino acids are conventional and are abbreviated as follows:

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when peptide sequences are presented as a series of one-letter and/or three-letter abbreviations, the sequences are presented in the amino to carboxy direction, in accordance with common practice.

“Diagnostically effective amount” is meant an amount of the substance in question which will, in a majority of patients, be an adequate quantity of substance to be able to detect the targeted tissue of cells if present in the patient to whom it is administered. The term “an effective amount” also implies that the substance is given in an amount which only causes mild or no adverse effects in the subject to whom it has been administered, or that the adverse effects may be tolerated from a medical and pharmaceutical point of view in the light of the severity of the disease for which the substance has been given.

“Integrated non-photoactive functional group” refers to a functional group within a bioactive molecule that does not exhibit a peak excitation and emission peak in the range of 350-1200 nm.

“Photoactive functional units” or “photoactive moieties” refers to any functional group or moiety exhibiting an absorption, excitation, and emission maxima in the wavelength range of 350-1200 nm. Such functional groups or moieties include, but are not limited to, fluorophores, chromophores, photosensitizers, and photoreactive moieties, wherein “fluorophores,” “chromophores,” “photosensitizers,” and “photoreactive” moieties have meanings that are commonly understood in the art.

“Photoreactive moiety” refers to a moiety of a molecule, which, when excited with light of wavelength 350 to 1200 nm, undergoes photochemical reaction to generate reactive species capable of causing tissue damage.”

“Pseudopeptide” is a modified peptide sequence in which either a peptide bond or an amino acid side chain is locally modified.

“Therapeutically-effective amount” refers to the amount of each agent that will achieve the goal of improvement in pathological condition severity and the frequency of incidence over treatment of each agent by itself, while avoiding adverse side effects typically associated with alternative therapies.

“Treatment” refers to any process, action, application, therapy, or the like, wherein a subject, including a human being, is provided medical aid with the object of improving the subject's condition, directly or indirectly, or slowing the progression of a pathological condition in the subject.

When introducing elements of the present invention or the embodiment(s) thereof, the articles “a”, “an”, and “the” are intended to mean that there are one or more of the elements. The terms “comprising”, “including” and “having” are intended to be inclusive and mean that there may be additional elements other than the listed elements.

The following examples illustrate specific embodiments of the invention. As would be apparent to skilled artisans, various changes and modifications are possible and are contemplated within the scope of the invention described.

### Example 1

### Preparation of Integrated Photoactive ST Receptor Binding Peptide of Formulas 2-4

For the synthesis of integrated photoactive analogs that bind to ST receptors, the first cartridge contains the Wang resin conjugated with Fmoc-Asn at the carboxyl terminal. The amino acid cartridges 2-7 contain Fmoc-Cys(Acm), Fmoc-Gly, Fmoc-Ala, Fmoc-Glu(γ-O-t-Bu), Fmoc-Ala, and Fmoc-Pro respectively; and cartridges 9-18 contain Fmoc-Cys(Acm), Fmoc-Cys(Acm), Fmoc-Leu, Fmoc-Asp(β-O-t-Bu), Fmoc-Cys(Acm), Fmoc-Cys(Acm), Fmoc-Tyr(O-t-Bu), Fmoc-Phe, Fmoc-Thr(O-t-Bu), and Fmoc-Asn respectively. The eighth cartridge contains photoactive Fmoc-protected amino acid residues. The amino acid cartridges are placed on the peptide synthesizer and the peptide is synthesized from the C- to the N-terminal position. The coupling reaction is carried out in the presence of 2-(1H-benzotriazol-1-yl)-1,1,3,3-tetramethyluronium hexafluorophosphate (HBTU)/N-hydroxybenzotriazole (HOBt). The Fmoc protecting group is removed with 20% piperidine in dimethylformamide, and the product is separated from the solid support with a cleavage mixture containing trifluoroacetic acid:water:phenol:thioanisole (85:5:5:5). The cleavage reaction typically takes about 6 hours to complete. The peptide is precipitated with t-butyl methyl ether, purified by HPLC, and lyophilized.

### Example 2

### Preparation of Integrated Photoactive Tenascin C Binding Peptide of Formulas 6-8

For the synthesis of photoactive tenascin C binding peptides, the amino acid cartridges 2-12 contain Fmoc-Thr(O-t-Bu), Fmoc-Leu, Fmoc-Glu(γ-O-t-Bu), Fmoc-Ile, Fmoc-Gly, Fmoc-Asp(β-O-t-Bu), Fmoc-Ile, Fmoc-Glu(γ-O-t-Bu), Fmoc-Ala, Fmoc-Leu, Fmoc-Pro respectively. The first cartridge contains Wang resin conjugated to photoactive Fmoc-protected amino acid residues. The synthesis, cleavage, and purification of the peptide are carried out in the same manner as described in Example 1.

### Example 3

### Preparation of Integrated Photoactive Endometriotic Peptides of Formulas 10-12

For the synthesis of photoactive endometriotic peptide, the first cartridge contains the Wang resin conjugated with Fmoc-Leu at the carboxyl terminal. The amino acid cartridges 2 and 3 contain Fmoc-Lys(ε-t-Boc), and Fmoc-Met respectively; and cartridges 5-11 contain Fmoc-Val, Fmoc-Ser(O-t-Bu), Fmoc-Leu, Fmoc-Lys(ε-t-Boc), Fmoc-Ile, and Fmoc-Asn, and Fmoc-Ala respectively. The fourth cartridge contains photoactive Fmoc-protected amino acid residues. The synthesis, cleavage, and purification of the peptide are carried out in the same manner as described in Example 1.

### Example 4

### Preparation of Integrated Photoactive Leukemia Cell Binding Peptides of Formulas 14-16

For the synthesis of photoactive leukemia cell binding peptide, the first cartridge contains the Wang resin conjugated with Fmoc-Ser(O-t-Bu) at the carboxyl terminal. The amino acid cartridges 2 and 3 contain Fmoc-Arg(O-t-Bu) and Fmoc-Leu respectively; and cartridges 5-7 contain Fmoc-Phe, Fmoc-Phe, and Fmoc-Ser(O-t-Bu) respectively. The fourth cartridge contains photoactive Fmoc-protected amino acid residues. The synthesis, cleavage, and purification of the peptide are carried out in the same manner as described in Example 1.

### REFERENCES

Hassan, M.; Klaunberg, B. A. Biomedical applications of fluorescence imaging in vivo. Comparative Medicine 2004, 54(6), 635-644.
Licha, K.; Olbrich, C. Optical imaging in drug discovery and diagnostic applications. Advances in Drug Delivery Reviews 2005, 57(8), 1087-1108.
Shah, K.; Weissleder, R. Molecular optical imaging: applications leading to the development of present day therapeutics. NeuroRx 2005, 2(2), 215-225.
Vasquez, M. E. et al. 6-N,N-Dimethylamino-2,3-naphthalamide: A new environment-sensitive fluorescent probes in δ and μ-selective opioid peptides. Journal of Medicinal Chemistry 2006, 49, 3653-3658.
Solban N.; Ortel, B.; Pogue, B.; Hasan, T. Targeted optical imaging and photodynamic therapy. Ernst Schering Research Foundation Workshop 2005, 49, 229-258.
Jain, R. K. Barriers to Drug Delivery in Solid Tumors. Scientific American 1994, 271, 58-65.
Hunter, D. H., and Luyt L. G. Single isomer technetium-99m tamoxifen conjugates. Bioconjugate Chemistry 2000, 11, 175-181.
Rajagopalan, R. Nitrogen sulfur ligands as opiate receptor drug mimics. U.S. patent 1994: U.S. Pat. No. 5,330,737.
Rajagopalan, R. Metal containing steroid mimics and ligands useful in the preparation thereof. U.S. patent 1997: U.S. Pat. No. 5,602,236.
Hom, R. K.; Katzenellenbogen, J. A. Synthesis of oxorhenium(V) complex mimic of a steroidal estrogen. J. Org. Chem. 1997, 62, 6290-6297.
Skaddan, M. B.; Katzenellenbogen, J. A. Integrated oxorhenium(V) complexes as estrogen mimics. Bioconjugate. Chem. 1999, 10, 119-129.
Miyata, K., et al. Synthesis and properties of a new fluorescent bicyclic 4-N-carbamoyl-deoxycytidine derivative. Organic Letters 2008, 8(8), 1545-1548.
Muller et al. Eds, Medical Optical Tomography, SPIE Volume IS11, 1993.
Waldman, S. ST receptor binding compounds and methods of using the same U.S. patent 1996: U.S. Pat. No. 5,518,888.
Edelberg, J., Ballard, V. Restoring vascular Function. US Publication No. 20060172943; PCT WO2005009366.
Schneider, H. et al. A novel peptide, PLAEIDGIELTY, for the targeting of αᵥβ₁-integrins. FESB Letters 1998, 429(3), 269-273.
Nothnick, W. B. Therapeutic Targets for the Treatment of Endometriosis, Expert Opinion 2004, 8(5), 459-471.
Mayo, K. et al. Partial peptide mimetics and methods. PCT WO 03/070751; US Publication No. 20040053828.
Jaalouk, D. et al. Compositions and methods that related peptides that bind selectively to leukemia cells. PCT WO 2006010070.
A. R. Gennaro (Ed.). Remington: The Science and Practice of Pharmacy, 20^{th} Edition. Lippincott Williams & Wilkins: Baltimore, 2000.
Janoff, A. S. et al. Methods of preparing low-toxicity drug-lipid complexes. U.S. patent 2002: U.S. Pat. No. 6,406,713.
Braun-Falco et al. (Eds.). Liposome Dermatics. Griesbach Conference, Springer-Verlag: Berlin, 1992.
Lasic and Martin (Eds.). Stealth Liposomes. CRC Press: London, 1995.
Wentroup, C. et al. Synthesis of 1-azaazulene and benz[a]azulene by carbene rearrangement. J. Am. Chem. Soc. 1984, 106(12), 3705-3706.
Nozoe, T. et al. Some synthetic applications of 3-carboxy-4-carboxymethyltropolone. Tohoku Daigaku Hisui Yoeka Kagaku Kankyusho Hokoku 1961, 10, 199-211.
Schneider, F. Synthese des D,L-imidazolyl-glycine and einiger derivate. Hoppe-Seyler's Zeitxchrift Fuer Physiologie Chemie, 1961, 324, 206-210.

What is claimed is:

1-25. (canceled)

26. An integrated photoactive analog of a non-photoactive peptide or pseudopeptide, the integrated photoactive analog having a photoactive amino acid substituted for a non-photoactive amino acid, wherein the photoactive amino acid has a photoactive functional group of the formula 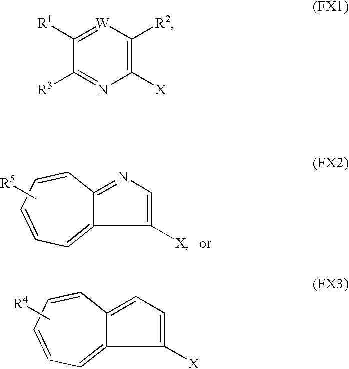
wherein:
R¹ to R³ are independently hydrogen, alkyl, aryl, —OR⁴, —SR⁵, —NR⁶R⁷, —CN, —CO₂R⁸, —NO₂, —COR⁹, —CNR¹⁰R¹¹, —SOR¹², or —SO₂R¹³;
W is N or —CR¹⁶;
X is —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, or —SO₂N(R²⁰)(CH₂)ₙ—;
R⁴ to R²⁰ are independently hydrogen, C₁-C₆ alkyl, C₁ to C₆ hydroxyalkyl, or C₁ to C₆ alkoxyalkyl;
n varies from 0 to 10; and
the non-photoactive amino acid is tyrosine, phenylalanine, glutamine or histidine when the photoactive functional group has formula (FX3).

27. The analog of claim 26, wherein the non-photoactive amino acid has a side chain having an aromatic or heteroaromatic moiety, and the photoactive amino acid has a side chain having an aromatic or heteroaromatic moiety having the same number of atoms in the ring structure as the aromatic or heteroaromatic moiety of the non-photoactive amino acid.

28. The analog of claim 26, wherein the non-photoactive amino acid is tyrosine, tryptophan, phenylanaline, histidine or glutamine.

29. The analog of claim 26, wherein the non-photoactive peptide or pseudopeptide has biological activity, and the analog retains the biological activity of the non-photoactive peptide or pseudopeptide.

30. The analog of claim 26, wherein the non-photoactive peptide or pseudopeptide has an ST receptor binding sequence, a tenascin C binding sequence, an endometriotic tissue binding sequence, or a leukemia cell binding sequence.

31. The analog of claim 26, wherein the photoactive amino acid comprises an azo group, diazo group, sulfanate group, thiadiazole group, a peroxide group, a phthalocyanine, a porphyrin, an extended porphyrin, or a benzopophyrin.

32. The analog of claim 31, wherein the non-photoactive peptide or pseudopeptide has biological activity, and substitution of the photoactive amino acid for the non-photoactive amino acid does not result in substantial loss of the biological activity.

33. The analog of claim 26, wherein the non-photoactive peptide or pseudopeptide comprises a sequence selected from SEQ ID NO 6, SEQ ID NO 10, SEQ ID NO 14 and SEQ ID NO 18.

34. An integrated photoactive analog of a non-photoactive peptide or pseudopeptide, the analog being of formula 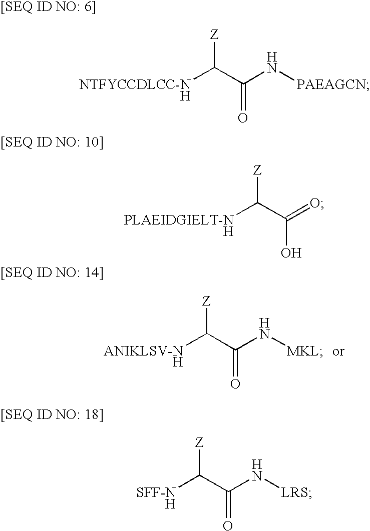
wherein Z has the formula: 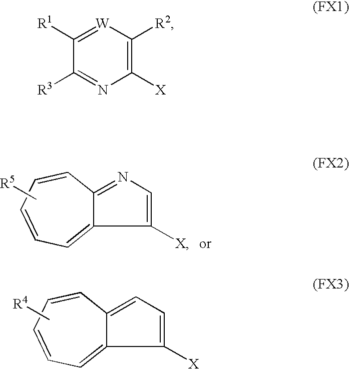
and wherein:
R¹ to R³ are independently hydrogen, alkyl, aryl, —OR⁴, —SR⁵, —NR⁶R⁷, —CN, 13 CO₂R⁸, —NO₂, —COR⁹, —CNR¹⁰R¹¹, —SOR¹², or —SO₂R¹³;
W is N or —CR¹⁶;
X is —(CH₂)ₙ—, —N(R¹⁷)CO(CH₂)ₙ—, —CON(R¹⁸)(CH₂)ₙ—, —N(R¹⁹)SO₂(CH₂)ₙ—, —NHCONH(CH₂)ₙ—, —O(CH₂)ₙ—, —CO₂(CH₂)ₙ—, —S(CH₂)ₙ—, —SO(CH₂)ₙ—, —SO₂(CH₂)ₙ—, or —SO₂N(R²⁰)(CH₂)ₙ—;
R⁴ to R²⁰ are independently hydrogen, C₁-C₆ alkyl, C₁ to C₆ hydroxyalkyl, or C₁ to C₆ alkoxyalkyl; and
n varies from 0 to 10.

35. An integrated photoactive analog of a non-photoactive peptide or pseudopeptide, the integrated photoactive analog comprising a peptide or pseudopeptide targeting group that targets a diseased tissue, cell, or receptor, and the integrated photoactive analog being of the following formula 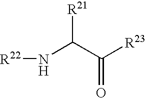
wherein:
R²¹ is a photoactive functional group;
R²² is hydrogen, an α-amino acid residue, or a sequence of two or more α-amino acid residues; and
R²³ is —OH, an α-amino acid residue, or a sequence of two or more α-amino acid residues.

36. The analog of claim 35, wherein 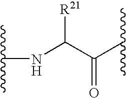
is a photoactive analog of a tyrosine, tryptophan, phenylalanine, or histidine residue.

37. The analog of claim 35, wherein the diseased tissue or cell is selected from cancerous tissue, leukemia cells, fibrotic epithelia, cystic fibrosis tissue, and endometriotic tissue.

38. The analog of claim 35, wherein the peptide or pseudopeptide targeting group comprises a ST receptor targeting group.

39. The analog of claim 35, wherein the peptide or pseudopeptide targeting group is a sequence selected from SEQ ID NO 7, SEQ ID NO 8, and SEQ ID NO 9.

40. The analog of claim 35, wherein the peptide or pseudopeptide targeting group comprises a tenascin C targeting group.

41. The analog of claim 35, wherein the peptide or pseudopeptide targeting group is a sequence selected from SEQ ID NO 11, SEQ ID NO 12, and SEQ ID NO 13.

42. The analog of claim 35, wherein the peptide or pseudopeptide targeting group comprises an endometriotic targeting group.

43. The analog of claim 35, wherein the peptide or pseudopeptide targeting group is a sequence selected from SEQ ID NO 15, SEQ ID NO 16, and SEQ ID NO 17.

44. The analog of claim 35, wherein the peptide or pseudopeptide targeting group comprises a leukemia cell targeting group.

45. The analog of claim 35, wherein the peptide or pseudopeptide targeting group is a sequence selected from SEQ ID NO 19, SEQ ID NO 20, and SEQ ID NO 21.


SEQUENCE LISTING (21 sequences)

SEQ ID NO: 1 - protein, 12 aa, Artificial Sequence
  synthesized peptide
PLAEIDGIELTY

SEQ ID NO: 2 - protein, 11 aa, Artificial Sequence
  synthesized peptide
ANIKLSVQMKL

SEQ ID NO: 3 - protein, 6 aa, Artificial Sequence
  synthesized peptide
SVQMKL

SEQ ID NO: 4 - protein, 9 aa, Artificial Sequence
  synthesized peptide
IKLSVQMKL

SEQ ID NO: 5 - protein, 10 aa, Artificial Sequence
  synthesized peptide
NIKLSVQMKL

SEQ ID NO: 6 - protein, 18 aa, Artificial Sequence
  synthesized peptide
NTFYCCDLCCYPAEAGCN

SEQ ID NO: 7 - protein, 18 aa, Artificial Sequence
  synthesized peptide
NTFYCCDLCCXPAEAGCN

SEQ ID NO: 8 - protein, 18 aa, Artificial Sequence
  synthesized peptide
NTFYCCDLCCXPAEAGCN

SEQ ID NO: 9 - protein, 18 aa, Artificial Sequence
  synthesized peptide
NTFYCCDLCCXPAEAGCN

SEQ ID NO: 10 - protein, 12 aa, Artificial Sequence
  synthesized peptide
PLAEIDGIELTY

SEQ ID NO: 11 - protein, 12 aa, Artificial Sequence
  synthesized peptide
PLAEIDGIELTX

SEQ ID NO: 12 - protein, 12 aa, Artificial Sequence
  synthesized peptide
PLAEIDGIELTX

SEQ ID NO: 13 - protein, 12 aa, Artificial Sequence
  synthesized peptide
PLAEIDGIELTX

SEQ ID NO: 14 - protein, 11 aa, Artificial Sequence
  synthesized peptide
ANIKLSVQMKL

SEQ ID NO: 15 - protein, 11 aa, Artificial Sequence
  synthesized peptide
ANIKLSVXMKL

SEQ ID NO: 16 - protein, 11 aa, Artificial Sequence
  synthesized peptide
ANIKLSVXMKL

SEQ ID NO: 17 - protein, 11 aa, Artificial Sequence
  synthesized peptide
ANIKLSVXMKL

SEQ ID NO: 18 - protein, 7 aa, Artificial Sequence
  synthesized peptide
SFFYLRS

SEQ ID NO: 19 - protein, 7 aa, Artificial Sequence
  synthesized peptide
SFFXLRS

SEQ ID NO: 20 - protein, 7 aa, Artificial Sequence
  synthesized peptide
SFFXLRS

SEQ ID NO: 21 - protein, 7 aa, Artificial Sequence
  synthesized peptide
SFFXLRS